(19) United States
(12) Patent Application Publication
(10) Pub. No.: US 2004/0220594 A1
(43) Pub. Date: Nov. 4, 2004

(54) Two-part clamping device

(75) Inventor: Bernard de Canniere, Brussels (BE)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Cardio Life Research S.A., Louvain-La-Neuve (BE)

(21) Appl. No.: 10/836,712

(22) Filed: Apr. 29, 2004

Related U.S. Application Data
(60) Provisional application No. 60/467,810, filed on May 2, 2003.

(51) Int. Cl. A61B 17/08

(52) U.S. Cl. 606/151; 606/157; 606/158

(57) ABSTRACT

A surgical clamping device for hollow anatomical structures such as, essentially, blood vessels, but also for tracheas, intestines, etc.

This device comprises two jaws, each jaw being formed of an elongate member having a distal end and a proximal end, and a mandrel that can be slipped over the two proximal ends of the two jaws and which is able to bring the axes of the two jaws closer together thus pinching an anatomical structure. The mandrel comprises a distal, pinching part and a proximal, handling part, these two parts being joint together by a locking part, the jaws being bendable at the level of the locking part when the clamp is activated, the distal, pinching part of the mandrel remaining in place by friction on the jaws when it is unlocked from the proximal, handling part.

This surgical clamping device applies more particularly to the clamping of the aorta.

The invention relates to clamping devices for hollow tubular anatomical structures such as, essentially blood vessels, but also for tracheas, intestines, etc.

### TECHNICAL BACKGROUND OF THE INVENTION

Heart surgery usually requires stopping the heart so as to obtain a stationary and exsanguinous operating site allowing a precise and delicate surgical operation.

Sternotomy is a destructive surgical approach that carries significant post-operative risks to the patient.

Furthermore, clamping the aorta is an operation which is considered to be delicate and high risk because, in particular, of the proximity of the pulmonary artery, the texture of which is known to be extremely fragile.

What is more, clipping the artery using a conventional clamp is a source of embolism of atheromatous material which, in most cases, lines the internal wall of the vessel.

For many years, heart surgery has been developing alternative techniques aimed at being less aggressive toward the patient. Doing away with the sternotomy is one of these approaches. In this case, the operation is carried out using mini-incisions allowing endoscopic instruments to be introduced.

An alternative solution allowing a great amount of patients to undergo minimally invasive heart surgery in complete safety was developed by the same inventor and disclosed in PCT/BE01/00211

In PCT/BE01/00211 was disclosed a surgical clamp which comprised a flexible guide; two jaws each formed of an elongate member pierced with a longitudinal canal and having a distal end and a proximal end. Said jaws were so designed that they could be slipped over the guide with their proximal end facing toward each of the ends of the guide; a mandrel that can be slipped over the two ends of the guide and over the jaws and which is able to bring the axes of the two jaws closer together and thus perform pinching.

The technique developed in PCT/BE 01 00211 now begins to be applied by numerous surgeons. However, the practice soon proved that slight changes in the design could improve dramatically the efficiency of the basic clamp.

In micro-surgery, owing to the restricted space the surgeon has to deal with, it is obvious that the operating field should be cleared out as quickly as possible of non-essential instruments. The clamp plays an essential role and has to be put in place at the very beginning of the operation and remains in place for a long time. The problem was to render it as little cumbersome as possible.

The non-obvious solution that was found was to manufacture the clamp in two parts, so that only an active, pinching head thereof remains in place, its proximal, handle part being removed from the way.

The advantage of the clamp of the invention is that the clamping remains effective and powerful through a minimal incision.

The device of the invention also applies particularly advantageously to the clamping of the aorta by using the anatomical space of Theile's transverse sinus as a natural guide, but it further can be used without guides in operations wherein the structure that has to be clamped is easy to reach.

Another advantage is that the risk of damaging an adjacent organ is reduced to a minimum, the disturbing proximal part being pulled aside and interfering thus no more with other instruments used by the surgeon.

The clamp of the invention can be used with equal ease for intra-thoracic and extra-thoracic vascular structures and for other anatomical structures including, in particular, the intestines. It may also be used as forceps for manipulating bones.

Other particulars and advantages of the invention will become apparent from the description hereinafter of some particular embodiments of the invention, reference being made to the appended drawings in which:

FIG. 1 is a perspective schematic view of the clamping of a human heart in the case of an operation with a sternotomy.

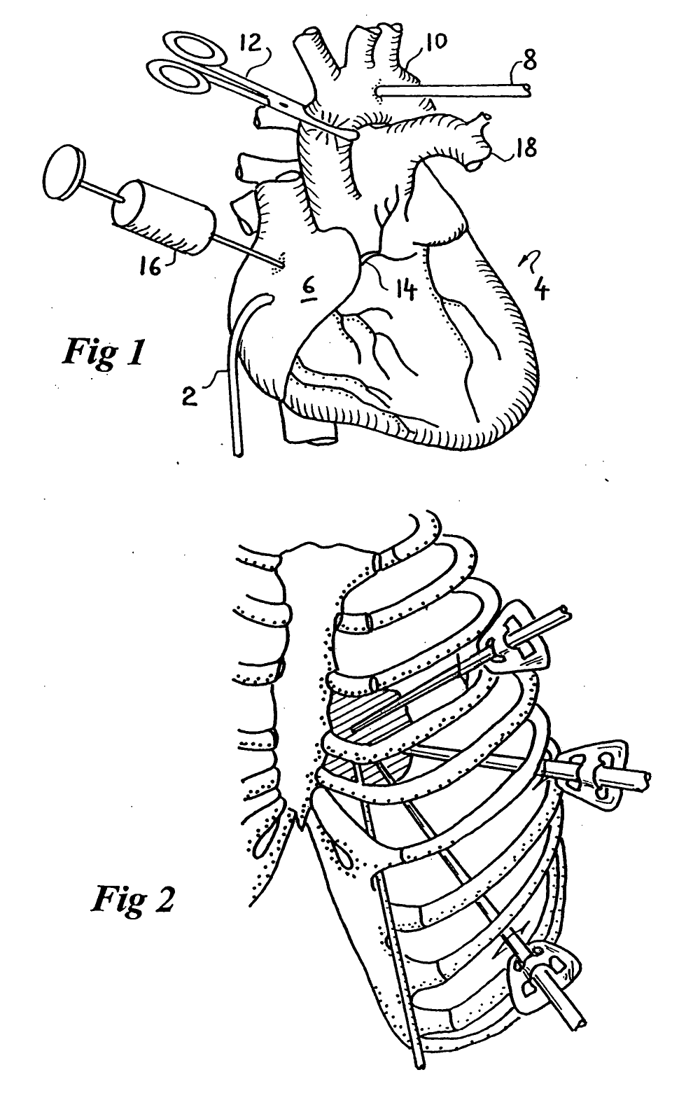

FIG. 2 is a view with cutaway of a heart operation with a mini-intercostal incision.

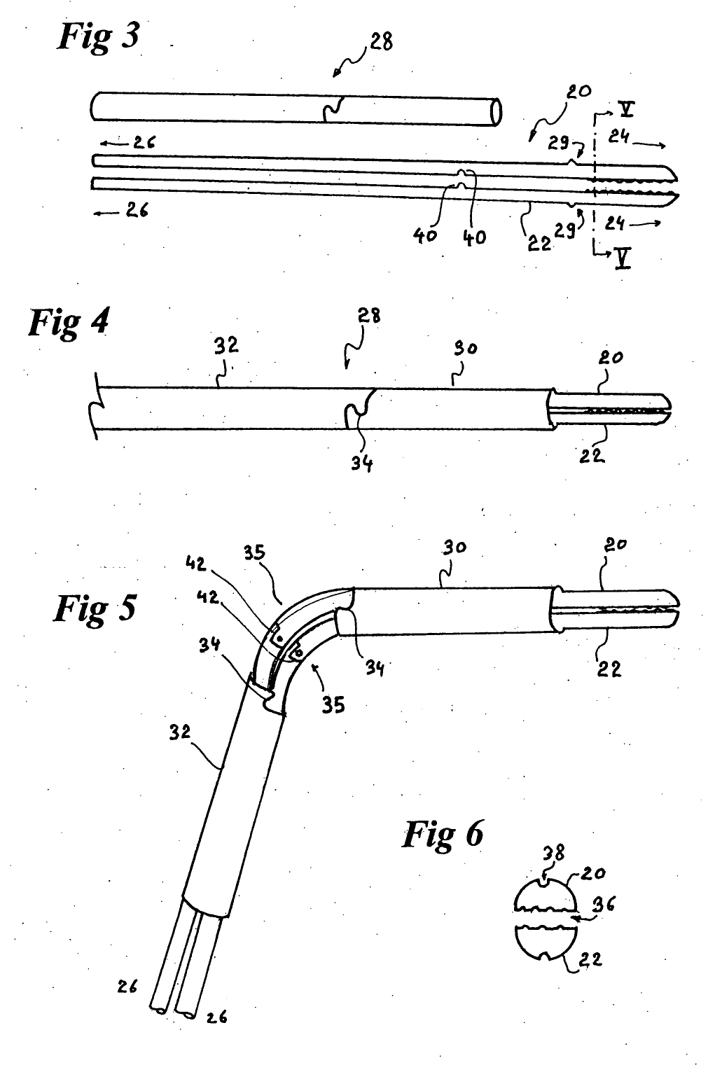

FIG. 3 is an exploded view of one form of embodiment of the clamp of the invention.

FIG. 4 is view in perspective of the clamp of FIG. 3 in active, pinching position,

FIG. 5 is view in perspective of the clamp of FIG. 3 with unlocked mandrel parts.

FIG. 6 is a view in section of a pair of jaw elements of the clamp of the invention.

FIG. 1 shows the various operations in preparation for a conventional heart operation, so as to obtain an exsanguinous and stationary operation field.

Venous blood (low in oxygen) is diverted down a cannula 2 as it enters the heart 4 via the right atrium 6 toward a heart-lung machine (not depicted) which namely reoxygenates it and rids it of its CO₂. The artificially oxygenated blood is then returned by a second cannula 8 at the aorta 10 into the patient's arterial circuit, thus short-circuiting the heart 4 and the pulmonary circulation so as to allow the intra-cardiac or extra-cardiac operation to be performed.

The heart 4 can therefore be stopped in order to obtain an exsanguinous and stationary operating field.

The heart is conventionally stopped using two joint operations:

clamping the aorta;

injecting a cardioplegy solution into the coronary circulation.

Clamping the aorta 10 consists in blocking the vessel using external forceps 12 which are applied between the arterial cannula 8 of the extra-corporeal circulation and the orifice of the coronary arteries 14. This operation isolates the coronary circulation from the blood flow generated by the ECC.

A cardioplegy solution can then be injected by an injection member 16 into the coronary circulation to “paralyze” the heart 4 with a view to allowing the surgeon to operate more precisely then he could on moving anatomical structures.

FIG. 2 shows another known approach, in which a heart operation is conducted via one or more incisions of the order of one centimeter long, allowing endoscopic instruments to be introduced.

The problem that arises in this case is that of reliably interrupting the circulation of the blood through the aorta, while at the same time avoiding the disadvantages inherent to introducing a balloon.

As was stated earlier, it is impossible to resort to conventional forceps in which the size of the jaws and their travel are out of proportion with the size of the intercostal incisions made.

The major advantage of the system according to the invention is that it allows the clamping to be performed without opening up the thorax but, what is more, with a lower risk of trauma to the pulmonary artery and of embolism.

The clamping device of the invention and its various components will be described with reference to the succession of FIGS. 3 to 5.

Two semi-rigid jaws 20,22, each formed of an elongate member having a distal, pinching end 24 and a proximal, handling end 26 are placed so that their distal ends position themselves one on each side of a vessel, organ or structure to be clamped. A hollow mandrel 28 is then slipped around the proximal ends of both jaws 20,22 and pulled along the jaws toward their distal ends. The jaws 20, 22 then each align with the axis of the mandrel 28 and move closer together, causing gentle and gradual pinching of the vessel, as can be seen on FIG. 4. Abutment means 29 placed on the jaws 20,22 limit the movement of the mandrel, so as not to bruise the vessel.

As described in PCT/BE01/00211, when the vessel is uneasy to reach, the jaws and the mandrel may be slipped over a guide inserted beforehand around the vessel.

One main innovative part of the present invention is that the mandrel 28 itself comprises two parts: a distal part 30 and a proximal, handling part 32, both parts being locked together by a locking fitting 34, here in the form of a bayonet.

When the organ or structure is firmly held between the jaws, the operator unlocks the locking fitting 34, here by simple twisting. The proximal, handling part 32 of the mandrel 28 may then be slightly pulled back (see FIG. 5) and the jaws 20,22 comprising a flexible section 35 at the level or upstream of the locking fitting 34, the proximal part of the whole device can be pulled aside, disencumbering the operating field for the surgeon.

At the end of the intervention, the two parts of the device are lined up, re-locked together and the mandrel 28 is pulled back as a whole, causing the jaws to relax the clamped structure, organ or vessel.

FIG. 6 shows, in cross section, the distal part of the jaws 20,22. In this embodiment, these may be provided with flexible jaw elements or with ribbings so as to spread the pressure over the organ which has been grasped. This section also displays a longitudinal groove 38 allowing a guide to be passed along the jaws.

Each member forming a jaw 30 is made of plastic. The faces of these jaws which face toward each other and are slightly toothed, form the jaw elements 36. The back of each jaw 20, 22, comprises a longitudinal groove 38 intended to accommodate a flexible guide.

Each of the jaw carries, at its central part, alignment means 40 which are intended to engage in the corresponding parts of the other jaw so as to prevent any relative movement of the jaws once their ends are inserted in the mandrel 28.

According to a form of embodiment, the mandrel 28 can be formed of a metal part bent over on itself.

To further improve the ease of use of the clamp, the jaws themselves may be provided with an unlocking feature 42, allowing a complete separation of the head part and the handling part, thus further disencumbering the operating field. As can be seen at FIG. 5, the unlocking feature can be, e.g., a combination of button and buttonhole or similar.

1. A surgical clamp comprising
two jaws, each jaw being formed of an elongate member extending along an axis having a distal end and a proximal end,
a mandrel that is able to be slipped over the proximal ends of both jaws and which is able to bring the axes of the two jaws closer together wherein the mandrel comprises a distal, pinching part and a proximal, handling part, these two parts being joint together by a locking part, the jaws being bendable at the level or upstream of the locking part when the clamp is activated, the distal, pinching part of the mandrel remaining in place by friction on the jaws when it is unlocked from the proximal, handling part.

2. A surgical clamp according to claim 1 wherein each jaw comprises a longitudinal canal for inserting a flexible guide.

3. A surgical clamp according to claim 1 having a slightly cylindrical section when assembled.

4. A surgical clamp according to claim 2 having a slightly cylindrical section when assembled.

5. The surgical clamp as claimed in claim 1, wherein the jaws have a malleable section at their proximal end.

6. The surgical clamp as claimed in claim 1, wherein the mandrel comprises a single longitudinal canal, the distal end of this canal being shaped in such a way as to cause the axes of the two jaws to be moved closer together by relative displacement.

7. The surgical clamp as claimed in claim 1, wherein the jaws are provided with flexible jaw elements near their distal end.

8. The surgical clamp as claimed in claim 1, wherein the jaws comprise abutment means for limiting the longitudinal movement of the mandrel

9. The surgical clamp as claimed in claim 1, wherein the proximal part of the jaws is equipped with ribbing collaborating with a mechanism supported by the mandrel.

10. The surgical clamp as claimed in claim 9, wherein the proximal part of the jaws is equipped with ribbing collaborating with a mechanism supported by the mandrel.

11. The surgical clamp as claimed in claim 1, wherein the jaws further comprise unlocking means.

12. The surgical clamp as claimed in claim 2, wherein the jaws further comprise unlocking means.
